(19) United States
(12) Patent Application Publication
(10) Pub. No.: US 2025/0061418 A1
(43) Pub. Date: Feb. 20, 2025

(54) RECEPTACLE CONTENT SENSING

(71) Applicant: CareFusion 303, Inc., San Diego, CA (US)

(72) Inventors: Ramkumar SUBRAMANIAN, San Diego, CA (US); Daniel M. ABAL, San Diego, CA (US); Brendan John BURGESS, Poway, CA (US); Rui TANG, San Diego, CA (US)

(21) Appl. No.: 18/723,834

(22) PCT Filed: Dec. 20, 2022
(86) PCT No.: PCT/US2022/053567

§ 371 Date: Jun. 24, 2024

Related U.S. Application Data
(60) Provisional application No. 63/294,060, filed on Dec. 27, 2021.

(51) Int. Cl. G06Q 10/087; G01F 23/20; G01F 23/292; G01F 23/296

(52) U.S. Cl. CPC G06Q 10/087; G01F 23/20; G01F 23/292; G01F 23/296

(57) ABSTRACT

A dispensing device monitors a plurality of containers for a change event, including at least one of: a fill order or a dispense of an item unit stored within the container, an opening or closing of the container, sensing a user within a predetermined proximity of the container, or a predetermined time. Based on an occurrence of the change event, the device causes an emission of a non-radio based transmission of one or more waves to an internal space of the container. A sensor with the container measures the one or more waves, and a supply level of an item unit within the container is determined based on the measurement and training data pertaining to measurements associated with past supply levels for the container. A notification is generated when the supply level satisfies a predetermined threshold.

### TECHNICAL FIELD

The present disclosure generally relates to the tracking inventory within closed containers.

### BACKGROUND

Centralized inventory systems are frequently used in the medical community to track and dispense medical products such as medications, medical devices, etc. Some of these items may be sophisticated and expensive instruments, and keeping close track of these items is desirable from a caregiver viewpoint as well as an ownership viewpoint. For a caregiver, it is not only important to be able to quickly and accurately locate where a needed item is stored, but also to be informed of inventory status. As an owner of the facility, it is important that very expensive items, such as electronic instruments, be protected from theft to the extent possible.

In such a centralized inventory system, medical products are stored in a storage area, such as a wall cabinet or other secure location. The dispensation of the products from the storage area may be tracked by requiring authorized users to indicate in a tracking log which products, and what quantity thereof, they have removed from the storage area. Similarly, low-stock and out-of-stock warnings may be provided by an alert user who notes the dwindling supply of a product as he removes it. These systems, however, rely upon the compliance of the users to track the dispensation of products therefrom, and are therefore prone to error. For example, current methods employ manual counting of inventory within medical containers at the end of each shift or at the end of the week. Some systems have used software to automatically update a current count when the container is accessed. However, software cannot account for discrepancies due to human error, and human counting is again required to resolve the discrepancies.

### SUMMARY

The subject technology described herein addresses the foregoing problems by automatically determining an estimate of container contents based on analyzing optical and acoustic signals associated with the container in accordance with predetermine trigger/change events. As user compliance is not required to conduct this analysis, the accuracy and efficacy of the dispensation tracking is improved. Moreover, as an accurate inventory can be reliably and automatically maintained, automated inventory warnings can be generated when warranted.

In this regard, a method includes monitoring each of a plurality of containers for a change event associated with the container, the change event comprising an indication of at least one of: a fill order for the container, a dispense of an item unit stored within the container, an opening or closing of the container, sensing a user within a predetermined proximity of the container, or a predetermined time; based on an occurrence of the change event: emitting a non-radio based transmission of one or more waves to an internal space within the container and for receipt by a sensor associated with the container, wherein the internal space is fashioned to store one or more item units; receiving a measurement of the one or more waves from the sensor; determining a supply level of an item unit within the container based on the measurement and training data pertaining to measurements associated with past supply levels for the container; and generating, when the supply level satisfies a predetermined threshold, a notification pertaining to the container and the supply level. Other aspects include corresponding systems, apparatuses, and computer program products for implementation of the foregoing features.

A disclosed system includes one or more containers each configured to store a supply of an item unit; one or more sensors, each configured to measure waves transmitted to an internal space of a container of the one or more containers, wherein the internal space of the container is fashioned to store one or more item units; one or more emitters, each configured to emit a non-radio based transmission of one or more waves to the internal space within for receipt by a respective sensor; and one or more processors configured to: monitor for a change event associated with a respective container, the change event comprising an indication of at least one of: a fill order for the respective container, a dispense of an item unit stored within the respective container, an opening or closing of the respective container, sensing a user within a predetermined proximity of the respective container, or a predetermined time; and based on an occurrence of the change event: cause a respective emitter to emit the non-radio based transmission of the one or more waves to an internal space within the respective container; receive a measurement of the wave from a sensor associated with the respective container; determine a supply level of an item unit within the respective container based on the measurement and training data pertaining to measurements associated with past supply levels for the respective container; and generate, when the supply level satisfies a predetermined threshold, a notification pertaining to the respective container and the supply level. Other aspects include corresponding methods, apparatuses, and computer program products for implementation of the foregoing features.

### BRIEF DESCRIPTION OF THE FIGURES

The accompanying drawings, which are included to provide further understanding and are incorporated in and constitute a part of this specification, illustrate disclosed implementations and together with the description serve to explain the principles of the disclosed implementations. In the drawings:

FIG. 1. depicts an example dispensing system, according to various aspects of the subject technology.

FIG. 2 depicts an open drawer of the dispensing system of FIG. 1 with receptacles contained therein, according to various aspects of the subject technology.

FIG. 3 depicts an exemplary portable smart carrier, according to various aspects of the present disclosure.

FIG. 4 is an example illustration of a receptacle, according to various aspects of the subject disclosure.

FIGS. 5A and 5B depict example containers configured for optical detection of contents, according to various aspects of the subject technology.

FIG. 6 depicts an example distance profile for optical detection of container contents, according to various aspects of the subject technology.

FIG. 7 depicts an example container configured for acoustic detection of contents, according to various aspects of the subject technology.

FIG. 8A depicts an example acquired voltage waveform and its corresponding frequency spectrum for an acoustic wave propagating through a container, according to various aspects of the subject technology. FIG. 8B depicts example resonance frequencies corresponding to empty, half-loaded, and full-loaded container.

FIGS. 8C, 8D, and 8E depict example acquired voltage waveforms and corresponding frequency spectrums for an oscillating piezoelectric circuit, according to various aspects of the subject technology.

FIG. 9 depicts an example process for using non-radio waves to determine the quantity of a container's contents, according to aspects of the subject technology.

FIG. 10 is a block diagram illustrating an example system for processing data to determine a status for a receptacle, according to aspects of the subject technology.

FIG. 11 is a conceptual diagram illustrating an example electronic system for using non-radio waves to determine the quantity of a container's contents, according to aspects of the subject technology.

### DESCRIPTION

In the following detailed description, numerous specific details are set forth to provide a full understanding of the present disclosure. It will be apparent, however, to one ordinarily skilled in the art that implementations of the present disclosure may be practiced without some of the specific details. In other instances, well-known structures and techniques have not been shown in detail so as not to obscure the disclosure.

Maintaining an accurate inventory of medical products (e.g., medications, medical devices, biological samples, donated organs, etc.) is an important part of providing exemplary patient care. Accordingly, the subject technology provides reliable, automated methods and systems for estimating an inventory of medical products from their respective storage devices (e.g., cabinets, stock rooms, drawers, etc.).

The subject technology provides for electro-mechanical monitoring of a container for a change event associated with the container. For example, a change event may be triggered when the container is opened or closed, or a dispensing cabinet which includes the container receives a dispense or fill request for a medication within the container. When the change request occurs, a non-radio based transmission of one or more waves is emitted into an internal space within the container, for receipt by a sensor associated with the container. For the purpose of this disclosure, non-radio based waves include, for example, acoustic and light energy and signals but do not include radio waves generated artificially by a transmitter and received by a receiver using an antennae. The sensor measures the one or more waves, and a supply level of an item unit within the container is determined based on the measurement and training data pertaining to measurements associated with past supply levels for the container. When the supply level satisfies a predetermined threshold, a notification pertaining to the container and the supply level may be generated.

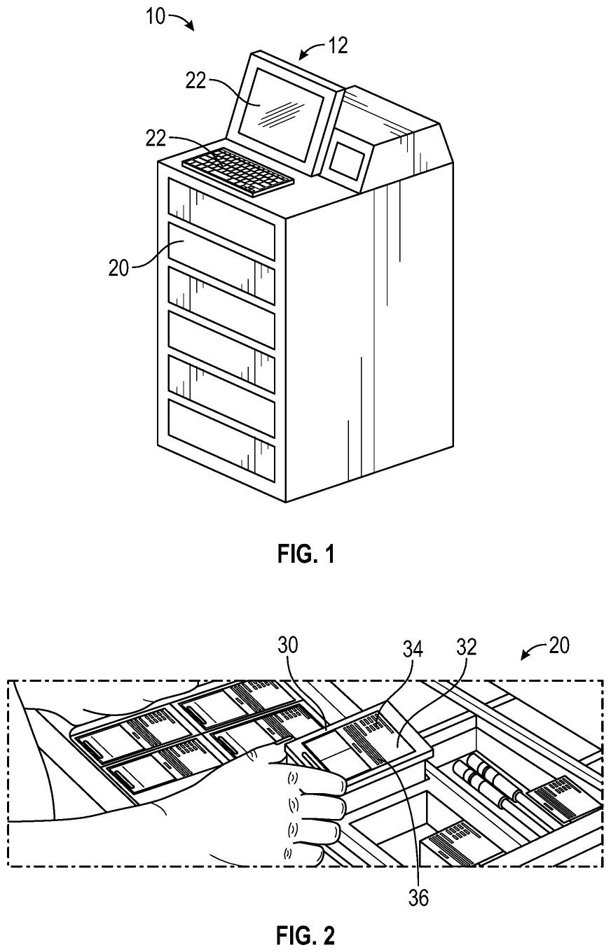

FIG. 1. depicts an example dispensing system 10, according to various aspects of the subject technology. This system 10 includes an automated dispensing machine (ADM) 12 with a plurality of drawers 20. In some implementations, the drawers 20 are secured such that the contents are not available for removal without authorization. The ADM 12 includes a user interface 22 and a processor (not visible in FIG. 1) that accepts input from the user through the user interface 22. An example dispensing process would begin with a user providing the credentials to the ADM 12, for example by providing a login name and password through the user interface 22, or by scanning a radio frequency identification (RFID) device such as a badge with an RFID chip embedded therein, with a radio frequency identification (RFID) sensor 24 located on the ADM 12. The ADM processor verifies that the identified user is authorized to remove at least one item from the ADM 12, and activates an item-selection display. The user selects an item to be dispensed. The ADM processor verifies that the user is authorized to remove the selected item and unlocks the drawer 20 that contains the selected item. The user removes the item, closes the drawer 20, and logs out, whereupon the ADM 12 locks the opened drawer 20.

FIG. 2 depicts an open drawer 20 of the ADM 12 of FIG. 1 with receptacles 30 contained therein, according to various aspects of the subject technology. Receptacles 30 may be marked with a label 32, including text 34 that provides information regarding the medication contained in the receptacle 30, for example the medication name, dose, and expiration date. Label 32 may also include a barcode 36 that may contain a portion of the same information or additional information, such as a tracking number associated with the latest filling of this particular receptacle 30. In some implementations, receptacle 30 may include an RFID tag that stores and transmits the same information when scanned by an RFID scanner. When the receptacle 30 is plugged into the drawer 20, the ADM processor of the ADM 12 can communicate with the memory of the receptacle 30 and retrieve the identification number associated with this receptacle 30. In some systems, a central database (not shown in FIG. 2) contains a list of identification numbers and information regarding the contents of the respective receptacles 30 and the ADM 12 can communicate with this database to retrieve this information, or provide communications between the database and receptacle 30 when receptacle 30 is docked with a docking interface of drawer 20. Thus, plugging a receptacle 30 into the drawer 20 informs the processor of the ADM 12 of the contents of that receptacle 30.

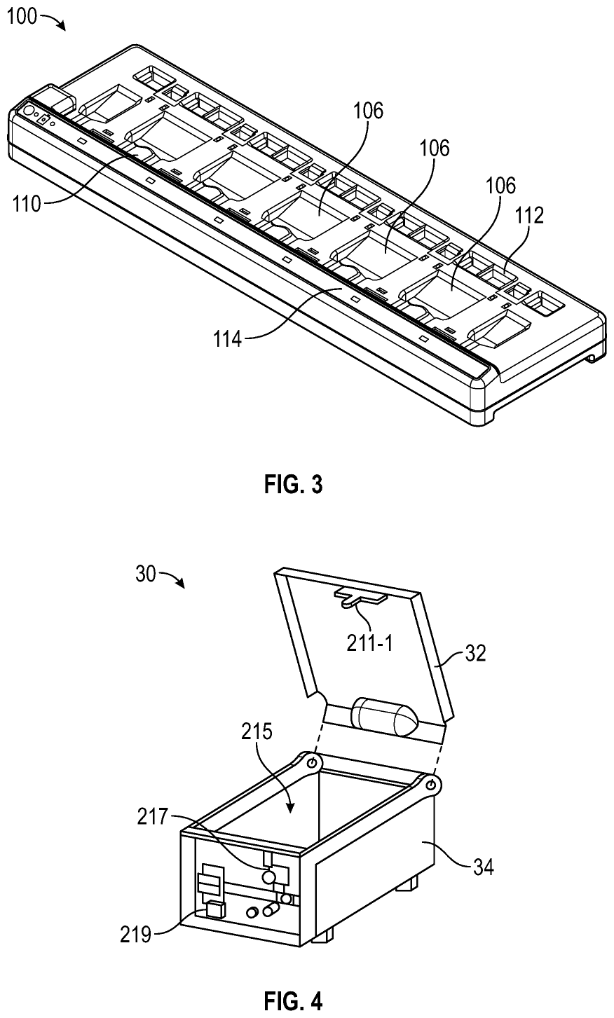

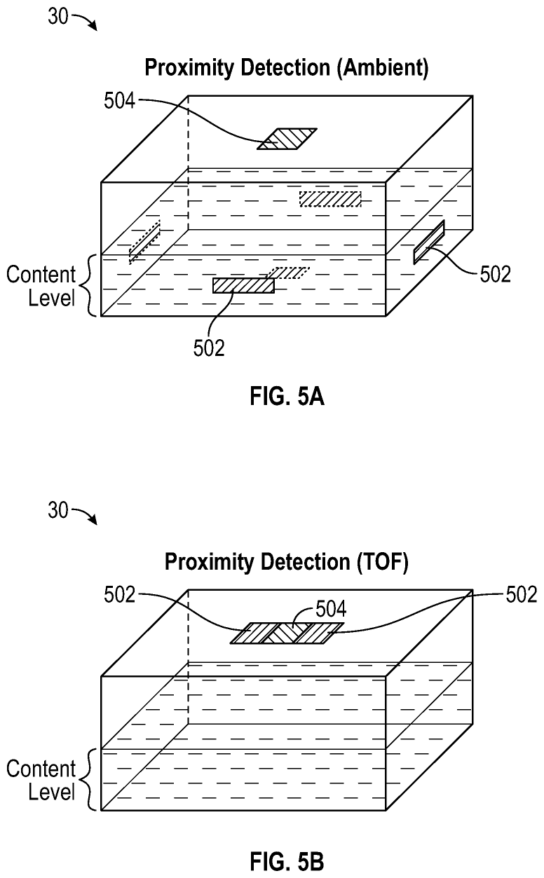

FIG. 3 depicts an exemplary portable smart carrier 100, according to various aspects of the present disclosure. This implementation of the smart carrier 100 comprises a body 102 with a handle 104, a plurality of docking locations 106, a processor 120 (not visible in FIG. 3, shown in FIG. 5), and an external connector 108. In general, the smart carrier 100 is sized to be portable and easily carried, for example by the handle 104, and transported, for example on a cart. Each of the docking locations 106 comprises a connector 110, a retention feature 112, and an indicator 114. In certain implementations, the connector 110 includes electrical contacts (not visible in FIG. 3) that may provide one or more of power, ground, and communication lines. In certain implementations, the retention feature 112 comprises a detent element (not visible in FIG. 3) that retains a latching feature of a receptacle (not shown in FIG. 3) until the applied removal force exceeds a predetermined value. In certain implementations, the retention feature 112 comprises a locking element (not visible in FIG. 3) that retains a latching feature of a receptacle (not shown in FIG. 3) until the locking element is unlocked by a command from the processor 120. In certain implementations, the indicator 114 comprises a visual indicator, for example a light-emitting diode (LED).

FIG. 4 is an example illustration of a receptacle 30, according to various aspects of the subject disclosure. In some implementations, the receptacle 30 is a secure receptacle 30, wherein the width and depth of the receptacles may vary. The receptacle 30 may include a lid 32 movably coupled to the body 34 and, in certain implementations, the lid 32 is hingedly attached to the body 34 at a back edge of the body 34.

A front cover has been removed from the body 34 to expose certain internal elements, including a processor 219 and a lid-securing actuator 217 that is configured to engage a latching feature such as the hook 211-1 of the lid 32. In some implementations, the lid-securing actuator 217 is configured to engage and retain the hook 211-1 so as to secure the lid 211 in a closed position. The processor 219 is operatively coupled to the lid-retaining actuator 217 and the interface connector 110 (not visible in FIG. 4). When the lid 211 is closed, the lid 211 cooperates with the body 213 to define a compartment 215. After the lid is closed, any items placed in the compartment 215 are secure until the receptacle 30 is docked with a receiving station such as a smart carrier 100 and a command is received by the processor 219 through the interface connector 210 to open the lid, whereupon the processor actuates the lid-retaining actuator 217 which releases the hook 211-1 and allows the lid 211 to open. In some implementations, the receptacle 201 may comprise a biasing element, such as a spring, that urges the lid 211 to move away from the closed position to an open position such that the lid 32 will self-open upon release of the hook 211-1 by the lid-securing actuator 217.

FIGS. 5A and 5B depict example containers configured for optical detection of contents, according to various aspects of the subject technology. In various implementations, subject technology uses an acoustic signal generated by a piezoelectric sensor and resonance detection algorithm to detect an amount of content inside a container (e.g., within the compartment of receptacle 30) in a non-invasive approach, and use that information to provide a closed loop inventor management of a certain medication or drug stored within the container, for example, in connection with a dispensing workflow.

In the example of FIG. 5A, optical sensors 502 are positioned on one or more walls (including, e.g., a bottom) of a compartment 215 within a receptacle 30. A corresponding light source 504 is positioned to emit light wave within the compartment 215. In the depicted example, the light source 504 is positioned within the compartment, under the lid 211. In some implementations, the light source may be positioned on a wall of the compartment or external to the compartment (e.g., projecting through an opening within a wall or lid of the compartment).

In some implementations, each sensor 502 may estimate and/or measure a distance between the sensor and contents within the container by measuring the amount of light reflected back from the object. In some implementations, the sensors include time-of-flight sensor. In other words, each sensor may measure the absolute distance (e.g., between the contents and the sensor) independent of target reflectance, based on the time the light takes to travel to the nearest object and reflect back to the sensor. While a light sensor 502 positioned on a bottom of the compartment may detect when no contents are present (at least over the sensor), the light sensors 502 positioned on the sides of the compartment may detect a distance between the side and contents within the compartment. The measurements may be cumulatively analyzed by a processor (e.g., processor 219) to provide an indication of a content level of the compartment (e.g., a number of items stored therein). In some implementations, the system determines whether the supply level of the container compartment is at, above, or below a predetermined level (e.g., empty, 25%, 50%, 75%, and 100%) based on the measurements.

In the example of FIG. 5B, the optical sensors 502 are positioned adjacent to the light source 502. In this regard, the subject technology may use optics based approach to identify if the container is full or empty, or at a certain threshold (e.g., 25%, 50%, 75%). As an alternative to or addition to acoustic sensors, the subject technology may employ optical sensors 502 in connection with a light source 504.

While optical sensors 502 and light source 504 are depicted separately, in some implementations, an optical sensor 502 and a light source 504 may be integrated into a single unit or chip. The optical sensors 502 may determine a relative height of the contents across the floor or bottom of the compartment by determining a distance from a roof or top of the compartment to a respective top of the contents. The processor (e.g., processor 219) may be configured to activate the sensors 504 and the light source 504 and to, based on comparing each measurement with one or more predetermined light wave amplitudes, determine a supply level of the container.

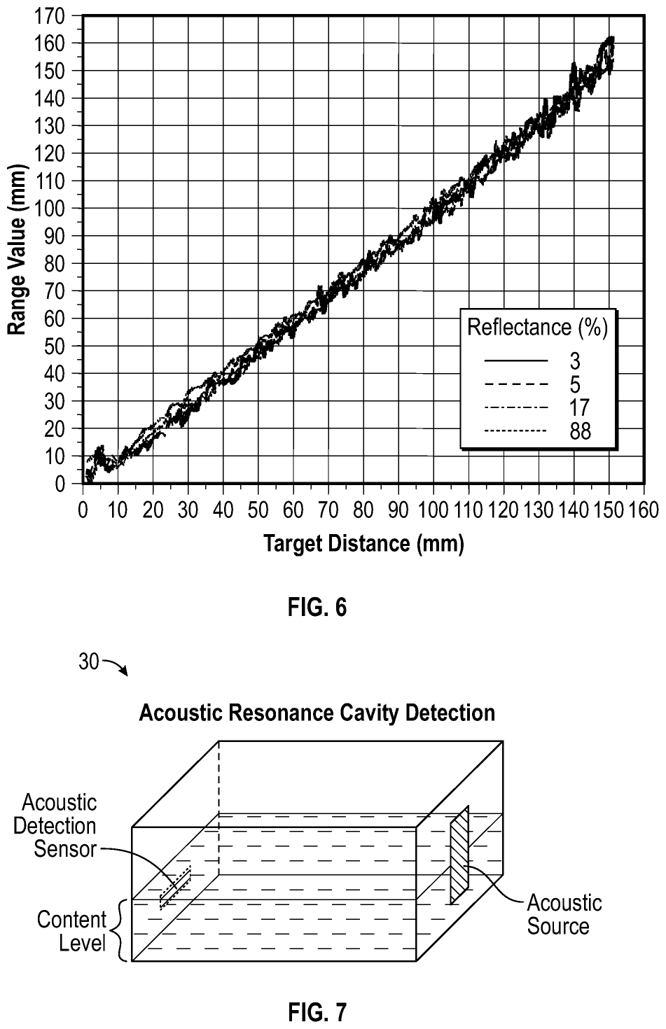

FIG. 6 depicts an example distance profile for optical detection of container contents, according to various aspects of the subject technology. In the depicted example, time of flight range values (vertical axis) is mapped to a distance between the emitter and sensor (horizontal axis) for measuring items of differing reflectance values (e.g., as a percentage). Training data may be generated by filing the container with various supply levels of an item and taking one or more measurements of the light from each sensor at each supply level. For randomized placement of items within the container, each measurement may correspond to a different orientation of the items. Each sensor (e.g., as shown in FIG. 5A or 5B) may be profiled for each location within the container. The training data may be provided as input to a machine learning algorithm which may then be used to determine, during operation, a supply level of the container based on current sensor readings.

FIG. 7 depicts an example container configured for acoustic detection of contents, according to various aspects of the subject technology. In the example of FIG. 7, one or more acoustic detection sensors 510 are positioned within or on the compartment 215, for example, on one or more walls or on the bottom or lid 211. A corresponding acoustic resonator 512 is positioned to emit an acoustic wave within the compartment 215. In the depicted example, the acoustic resonator is a piezoelectric diaphragm. However, the acoustic resonator 512 may be different vibration-powered generator such as a magnetic coil, or an ultrasonic transducer, or speaker driver. In some implementations, the acoustic resonator 512 may be positioned on a wall of the compartment or external to the compartment (e.g., causing the entire compartment or a portion of the compartment to resonate).

According to various implementations, a resonance frequency of the compartment 215 is used to detect a supply level of the contents (e.g., number of item units) within the compartment. In some implementations, the acoustic resonator 512 (e.g., a piezoelectric diaphragm) is used to detect the cavity resonance frequency of the compartment and a frequency shift under different supply levels. The resonance frequency is the frequency at which the sound of the container itself naturally amplifies. Placing objects with the container change the resonant frequency. The subject technology maps placement and number of objects within a container with resonant frequencies so that a current supply level of the container can be reliably estimated based on a current determination of the resonant frequency.

A piezo diaphragm within the compartment may be connected to an oscilloscope which acquires a voltage signal generated by the piezo diaphragm. When the receptacle or compartment is tapped, an acoustic wave propagates the compartment cavity with the resonance frequency component lasting for a shorter period of time (e.g., around 2 ms) while other frequency components decay rapidly. Accordingly, the frequency component may be detected based on the acquired voltage signal and observed based on fast Fourier transform spectrum analysis.

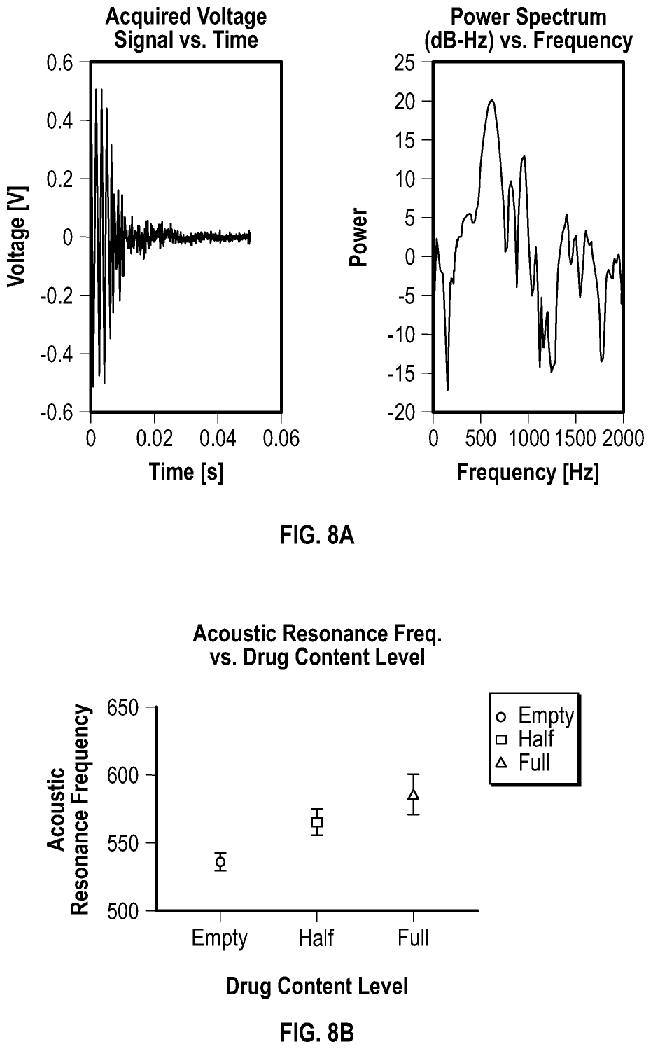

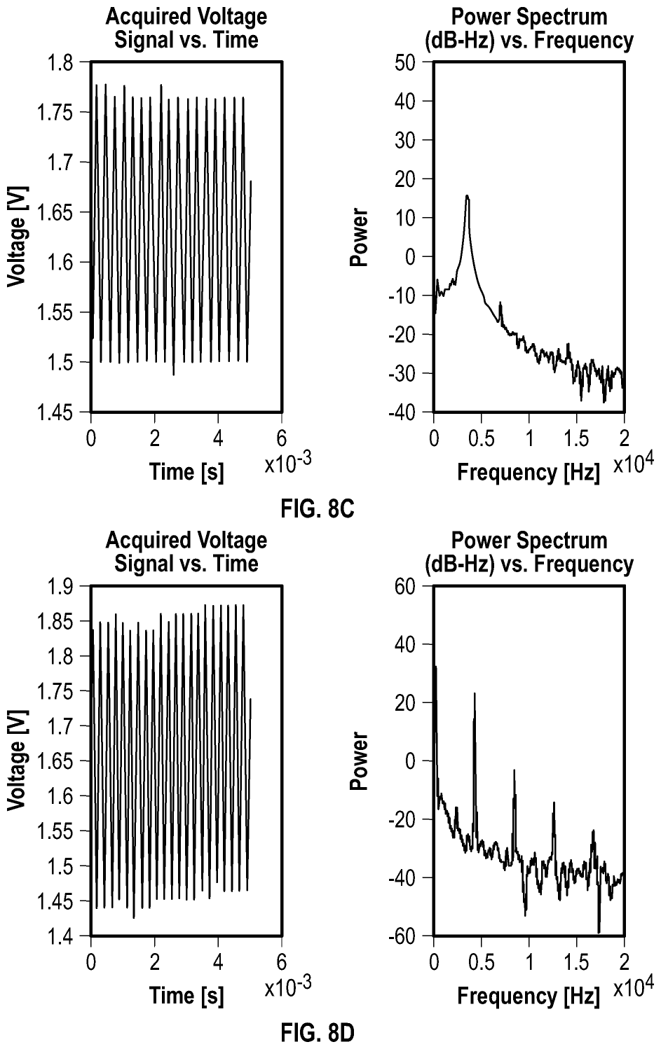

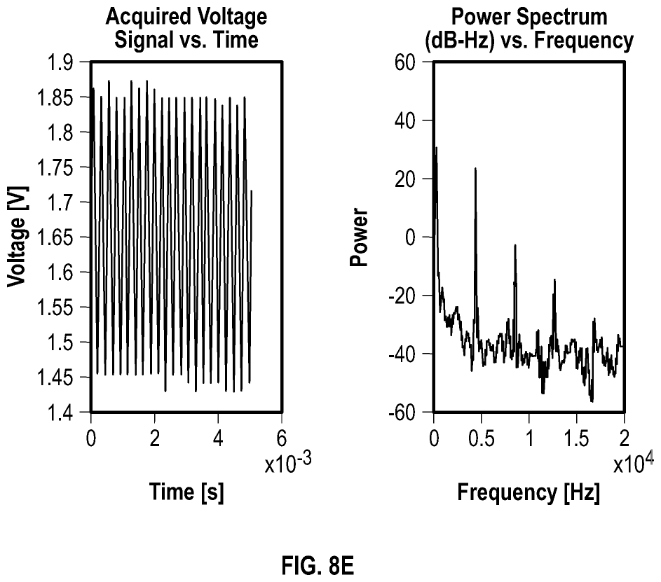

FIG. 8A depicts an example acquired voltage waveform and its corresponding frequency spectrum for an acoustic wave propagating through a container, according to various aspects of the subject technology. FIG. 8B depicts example resonance frequencies corresponding to empty, half-loaded, and full-loaded container. Tests of different of piezo diaphragm types found that a diaphragm with 4.2 k Hz resonance frequency exhibits a consistent response with different content inventory levels. A 20 Hz frequency shift was observed between empty, half and full inventory levels, as shown in the example graph of FIG. 8B. To generate training data for each inventory level, the test may be repeated multiple times (e.g., 20 times) to obtain a frequency response range.

Turning back to FIG. 7, in some implementations, the acoustic resonator 512 (e.g., a piezoelectric diaphragm) is triggered to oscillate at its resonant frequency using a waveform generator (e.g., integrated in or associated with processor 219), and a microphone (e.g., a MEMS microphone chip) may be used to receive the sound wave propagating across the container cavity, and observe the acquired voltage signal amplitude and frequency.

FIGS. 8C, 8D, and 8E depict example acquired voltage waveforms and corresponding frequency spectrums for an oscillating piezoelectric circuit, according to various aspects of the subject technology. The depicted waveforms resulted from testing 3.5 kHz, 3.9 kHz, and 4.2 kHz piezoelectric diaphragms with 2V peak-peak driving voltage a number of times. As shown by the depicted results, the receiving sound wave frequency remained unchanged while the signal voltage amplitude varied among different experiment runs.

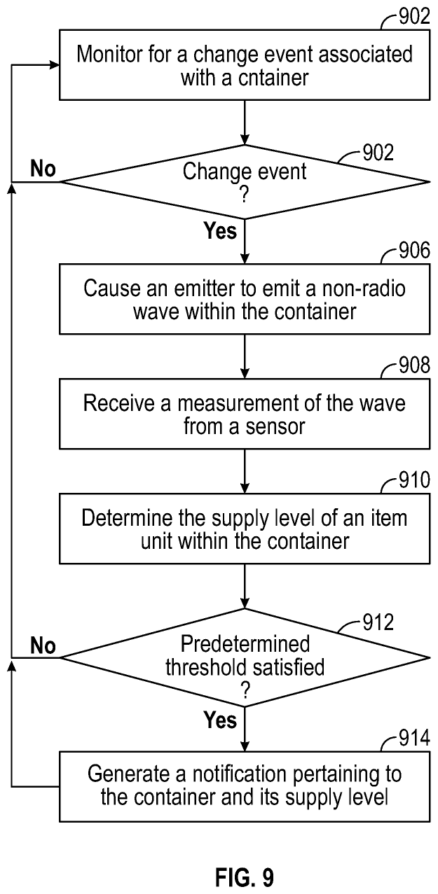

FIG. 9 depicts an example process for using non-radio waves to determine the quantity of a container's contents, according to various aspects of the subject technology. For explanatory purposes, the various blocks of example process 900 are described herein with reference to FIGS. 1-8, as well as the components and processes described herein. The one or more blocks of process 900 may be implemented, for example, by a system including, for example, automated dispensing machine (ADM) 12, carrier 100, or receptacle 30. In some implementations, one or more of the blocks may be implemented based on one or more machine learning algorithms. In some implementations, one or more of the blocks may be implemented apart from other blocks, and by one or more different processors or devices. Further, for explanatory purposes, the blocks of example process 900 are described as occurring in serial, or linearly. However, multiple blocks of example process 900 may occur in parallel. In addition, the blocks of example process 900 need not be performed in the order shown and one or more of the blocks of example process 900 need not be performed.

As described previously, the disclosed system may include one or more containers each configured to store a supply of an item unit, one or more sensors, and one or more emitters. Each sensor may be configured to measure waves transmitted to an internal space of a container. Each emitter may be configured to emit a non-radio based transmission of one or more waves to the internal space within for receipt by a respective sensor. In some implementations, the system includes automated dispensing machine (ADM) 12 with a plurality of drawers 20. A respective drawer may be configured to hold a plurality of the containers.

In the depicted example, the system monitors for a change event associated with a container (902). In an ADM 12, the system may be configured to monitor each of the drawers, or each of the containers within the drawers, for the change event. According to various implementations, the change event may include an indication of one or more of the following: (a) placement of a fill order for a container; (b) dispense of an item unit stored within a container; (c) opening or closing of a container; (d) sensing of a user within a predetermined proximity of a container; (e) passing of a predetermined amount of time; or (f) receipt of a signal respecting container.

Absent a change event, the system may continue to monitor for a change event associated with a container (904). However, once a change event occurs (904), the system causes a respective emitter to emit a transmission of non-radio wave to an internal space within the respective container (906). As described previously, the non-radio wave may be emitted from a location external the container, or from an emission device internal to the container. Following emission, the system will receive a measurement of the non-radio wave from at least one sensor associated with the respective container (908). As depicted in FIGS. 5A and 5B, the sensors may be placed on one or more walls of the container, and/or on a floor and/or lid of the container.

In some implementations, the emitter associated with the respective container induces an acoustic wave within the respective container. In such implementations, a respective sensor associated measures the frequency and/or amplitude of the acoustic wave. In some implementations, the emitter includes a light source to induce a light wave within the respective container for measurement by the sensor(s). In such implementations, each sensor works in conjunction with the emitter measure a distance between the light source and contents within the container (or to an opposite side of the container when no contents are within the light path).

After the system receives the measurement of the non-radio wave, it then determines a supply status (e.g., supply level; 25%, 50%, 75% of capacity, etc.) associated with an item unit within the respective container based on measurement and training data pertaining to measurements associated with past supply levels for the respective container (910). As described previously, the training data may have been previously generated by filing the container with various supply levels of an item and taking one or more sensor measurements (e.g., acoustic or light) at each supply level. A population of measurements may be recorded, each corresponding to a randomized placement of items within the container, and each measurement or range of measurements associate with each supply level recorded (including, e.g., orientation of the items and/or location of the sensor within the container). The training data may be provided as input to a machine learning algorithm which may then be used to determine, during operation, a supply level of the container based on the current sensor readings.

In some implementations, the training data includes an intended supply level or a geometry of an interior of the respective container. In implementations pertaining to acoustic measurements, the training data may indicate a resonance frequency of the respective container at different supply levels (e.g., no of items) stored in the container. In implementations pertaining to light measurements, the training data may include one or more predetermined light wave amplitudes associated with a distance between contents in the container and a wall, floor, or roof of the respective container.

In the foregoing implementations, the measurement(s) received from the sensor(s) are compared with the training data in order to determine the supply level of the item unit within the respective container.

Once a supply level of the item unit within the respective container is determined, the system then compares the supply level with a predetermined threshold (912). If the supply level does not satisfy the predetermined threshold (e.g., 25%, 50%, 60% of capacity), the system returns to monitoring for a change event associated with a container (902). However, if the supply level satisfies the predetermined threshold (e.g., below 25%, 50%, 60% of capacity), the system generates a notification pertaining to the respective container and the supply level (914). Then, the system may return to monitoring for a change event associated with a container (902).

According to various implementations, the notification may include a visual or auditory alert at a dispensing device associated with the container. In some implementations, the notification includes a replenishment request for additional supply of the item unit stored within the respective container. In such implementations, the system may be configured to send the notification to a server configured to facilitate replenishment of the item unit. In this regard, generating a notification may include: (a) sending a replenishment signal to an inventory system, requesting a restock of the item unit; (b) receiving an indication that an amount of the item unit was restocked within the respective container; and (c) updating the training data based on the determined supply level and the amount.

In some implementations, the system may indicate a secondary check on the supply level when the supply level satisfies the predetermined threshold. For example, the system may activate a load cell, which measures a weight associated with the supply level. The system may then determine whether the measured weight corresponds to the supply level determined by measurement of the non-radio waves.

Many of the above-described examples of FIGS. 1 to 9, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

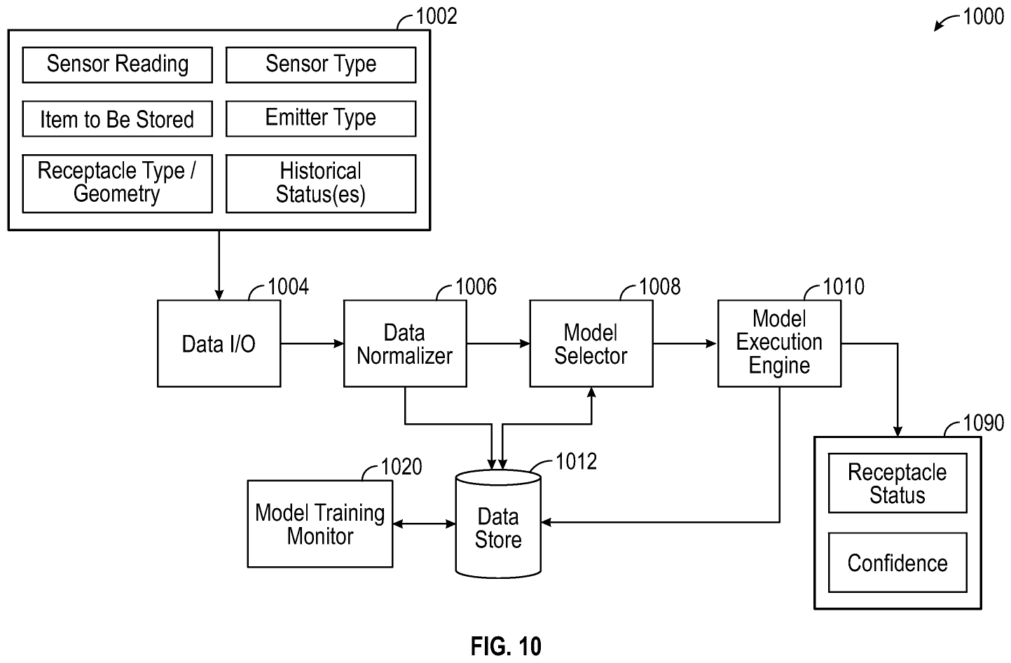

FIG. 10 is a block diagram illustrating an example system for processing data to determine a status for a receptacle. All or portions of the system 1000 may be implemented using one or more of the devices described. The processing of the system 1000 may include artificial intelligence or other operations whereby the system processes inputs and generates outputs with apparent intelligence. The artificial intelligence may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a medication station scanning status. In such instance, the training may include receiving the three characteristics as inputs to the model and adjusting the characteristics of the model such that for each set of three characteristics, the output device state matches the desired scanning status associated with the historical data. In this way, the model can output a value associated with the status of the medication station.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine-readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. For example, a deep scan threshold may be defined whereby after the threshold number of deep scans are performed, sufficient data is available to retrain the model. In some implementations, the retraining may be periodic, based on an elapsed time since the model was previously trained. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

Still referring to the system 1000 of FIG. 10, a data input/output (IO) may receive one input values 1002. The example input values 1002 shown in FIG. 10 include a sensor reading or measurement, information identifying a sensor or sensor type that generated the reading or measurement, information identifying item(s) to be stored or currently stored in a receptacle, information identifying the receptacle or geometry thereof, historical status information for the receptacle, or other information available to the system 1000.

A data normalizer 1006 may be included to prepare the input value(s) 1002 for analysis. The data normalizer 1006 may include encoding an input value 1002 to one of a known set of values. For example, the sensor type may be normalized to a value indicating whether the sensor is an optical or acoustic sensor. All or a portion of the normalized data and/or input value(s) 1002 may be stored in a data store 1012.

As used herein a “data store” may be implemented using hard disk drives, solid state memories and/or any other type of non-transitory computer-readable storage medium accessible to or by a device described. A data store may also or alternatively be distributed or partitioned across multiple local and/or remote storage devices as is known in the art without departing from the scope of the present disclosure. A data store may include or be embodied in a data storage web service or other network attached device configured to store data.

Normalized values or input values may then be provided to a model selector 1008. The model selector 1008 may be included to allow the system 1000 to dynamically adjust processing based on the received input values 1002. For example, the data store 1012 may include a catalog of trained models, indexed by one or more values. The model selector 1008 may transmit a query including one or more values from the input value(s) 1002 or normalized values to the data store 1012. The data store 1012 may then identify a trained model appropriate for processing the input values based on the query.

The system 1000 may include a model execution engine 1010. The model execution engine 1010 may be configured to process the input value(s) 1002 and/or normalized values using the identified trained model. Processing the input value(s) 1002 and/or normalized values may include providing the values or portion thereof as inputs to the trained model. The trained model, when executed by the model execution engine 1010, may provide one or more output values 1090. The output values may include an indicator of the status for the receptacle. Examples of the status include level of fullness, level of emptiness, estimated number of items, secured state (e.g., locked, unlocked, open), bin integrity (e.g., cracked), or other described herein. Some models may be trained to provide multiple statuses. Some models may include confidence values for each status. The confidence value may reflect a likelihood that the reported status is the actual status of the receptacle. The output values 1090 may be stored in the data store 1012.

The system 1000 may include a model training monitor 1020. The model training monitor 1020 may be configured to watch the data in a data store such as the data store 1012 and identify, based on the data, a need for retraining a model. For example, a threshold quantity of new data records may be defined after which, the model may need to be retrained to account for the new data. In some implementations, the model training monitor 1020 may be configured to identify a training need based on input values 1002 or other value available to the system 1000.

The features described may reference assessment of supply level. However, as discussed with reference to FIG. 10, supply level is one indicator of a status for a receptacle. The features may be applied to detect and respond to additional or alternative statuses. The features described may reference providing notifications in response to certain supply levels. However, the features may be applied to adjust the receptacle, a dispenser in which the receptacle is placed, or other device in communication with either. For example, if the supply level of a receptacle does not correspond to a threshold, access to the receptacle may be restricted such as to users with supervisory permissions. As another example, if the status indicates the receptacle is potentially damaged, the station may not permit access to the receptacle to receive items but only to dispense items. Other examples of adjustments that may be based on receptacle status include: locking a receptacle, unlocking a receptacle, changing permissions or roles for accessing a receptacle, changing a workflow for accessing a receptacle (e.g., require a second user to present their credentials to witness an access of the receptacle), actuate the receptacle (e.g., a receptacle may include or be coupled to a vibration device that can jostle items within the receptacle to give a different potentially more accurate measurement), change frequency of the non-radio emission for the receptacle, change timing of scanning for the receptacle, or the like.

As user herein, the terms “correspond” or “corresponding” when referring to values encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

The thresholds described may be static or dynamic. Static thresholds are predetermined thresholds that remain constant. Dynamic thresholds are changed throughout operation of the system based on changes to the system, a receptacle, or items stored therein. Whether static or dynamic, a threshold may be specified as a value or a range of values.

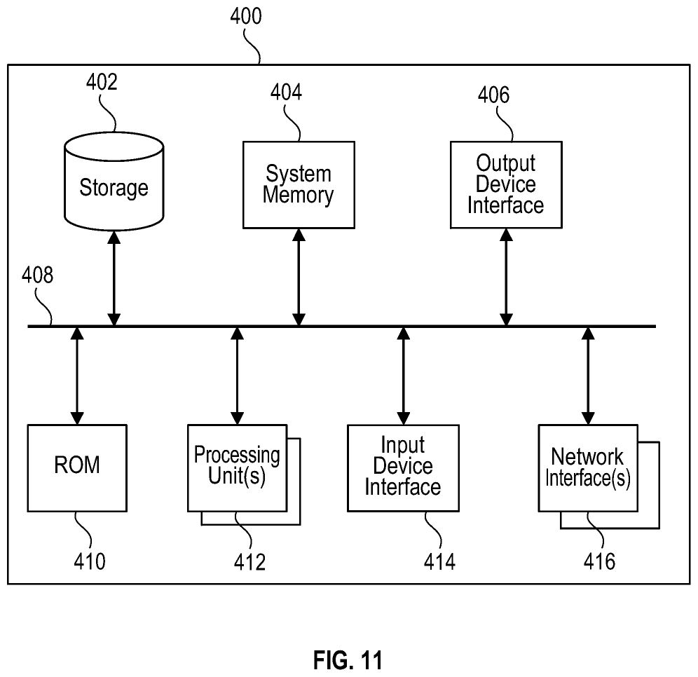

FIG. 11 is a conceptual diagram illustrating an example electronic system 400 for using non-radio waves to determine the quantity of a container's contents, according to aspects of the subject technology. Electronic system 400 may execute software instructions associated with one or more portions or steps of process 200, or components and processes provided by FIGS. 1-9. System 400 may be part of a receptacle 30, or associated with or include the processor of ADM 12, or processor 219 of receptacle 30 or of the carrier 100, or a computing device operably connected to any of the foregoing. Electronic system 400 may be representative, in combination with the disclosure regarding FIGS. 1-9. In this regard, electronic system 400 may be a PCBA, a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 614, an output device interface 406, and one or more network interfaces 416. In some implementations, system memory 404 and/or ROM 410 may be part of receptacle, as previously described. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such as a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 11, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

The subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A computer-implemented method, comprising: monitoring each of a plurality of containers for a change event associated with the container, the change event comprising an indication of at least one of: a fill order for the container, a dispense of an item unit stored within the container, an opening or closing of the container, sensing a user within a predetermined proximity of the container, or a predetermined time; based on an occurrence of the change event: emitting a non-radio based transmission of one or more waves to an internal space within the container and for receipt by a sensor associated with the container, wherein the internal space is fashioned to store one or more item units; receiving a measurement of the one or more waves from the sensor; determining a supply status of an item unit within the container based on the measurement and training data pertaining to measurements associated with past supply statuses for the container; and generating, when the supply status satisfies a predetermined status, a notification pertaining to the container and the supply status.

Clause 2. The computer-implemented method of Clause 1, wherein monitoring each of the plurality of containers comprises: monitoring each of a plurality of containers within a drawer of a dispensing device.

Clause 3. The computer-implemented method of Clause 1 or Clause 2, wherein the notification comprises a replenishment request for additional supply of the item unit stored within the container, the method further comprising: sending the replenishment request to a server configured to facilitate replenishment of the item unit.

Clause 4. The computer-implemented method of any one of Clauses 1 through 3, wherein the training data comprises an intended supply status and a geometry of an interior of the container.

Clause 5. The computer-implemented method of any one of Clauses 1 through 4, further comprising: identifying a plurality of resonance frequencies of the container at different supply levels from the training data; wherein emitting the non-radio based transmission of one or more waves to the internal space comprises inducing an acoustic wave within the container for measurement by the sensor; and comparing the measurement with one or more predetermined acoustic wave amplitudes and the plurality of resonant frequencies, wherein determining the supply status based on the measurement comprises determining the supply status based on the comparing.

Clause 6. The computer-implemented method of any one of Clauses 1 through 4, further comprising: identifying a light wave profile of the container from the training data; wherein emitting the non-radio based transmission of one or more waves to the internal space comprises inducing a light wave within the container for measurement by the sensor; comparing the measurement with one or more predetermined light wave amplitudes; and wherein determining the supply status based on the measurement comprises determining the supply status based on the comparing.

Clause 7. The computer-implemented method of any one of Clauses 1 through 6, further comprising: determining that the supply status satisfies the predetermined status; responsive to determining that the supply status satisfies the predetermined threshold: activating a load cell associated with the container to measure a weight associated with the determined supply status; measuring the weight associated with the determined supply status; and determining that the measured weight corresponds to the determined supply status.

Clause 8. The computer-implemented method of any one of Clauses 1 through 7, wherein generating the notification comprises: sending a replenishment signal to an inventory system, the replenishment signal requesting a restock of the item unit.

Clause 9. The computer-implemented method of Clause 8, further comprising: receiving an indication that an amount of the item unit was restocked within the container; and updating the training data based on the determined supply status and the amount.

Clause 10. The computer-implemented method of any one of Clauses 1 through 9, wherein generating the notification comprises: providing a visual or auditory alert at a dispensing device associated with the container.

Clause 11. A system, comprising: one or more containers each configured to store a supply of an item unit; one or more sensors, each configured to measure waves transmitted to an internal space of a container of the one or more containers, wherein the internal space of the container is fashioned to store one or more item units; one or more emitters, each configured to emit a non-radio based transmission of one or more waves to the internal space within for receipt by a respective sensor; and one or more processors configured to: monitor for a change event associated with a respective container, the change event comprising an indication of at least one of: a fill order for the respective container, a dispense of an item unit stored within the respective container, an opening or closing of the respective container, sensing a user within a predetermined proximity of the respective container, or a predetermined time; and based on an occurrence of the change event: cause a respective emitter to emit the non-radio based transmission of the one or more waves to an internal space within the respective container; receive a measurement of the wave from a sensor associated with the respective container; determine a supply status associated with an item unit within the respective container based on the measurement and training data pertaining to measurements associated with past supply statuses for the respective container; and generate, when the supply status satisfies a predetermined threshold, a notification pertaining to the respective container and the supply status.

Clause 12. The system of Clause 11, further comprising: a dispensing device comprising one or more drawers configured to store the one or more containers, wherein monitoring each of the one or more containers comprises monitoring each of a plurality of containers within the one or more drawers for the change event, and wherein the training data comprises an intended supply status and a geometry of an interior of the respective container.

Clause 13. The system of Clause 11 or Clause 12, wherein the notification comprises a replenishment request for additional supply of the item unit stored within the respective container, the one or more processors further configured to: send the replenishment request to a server configured to facilitate replenishment of the item unit.

Clause 14. The system of any one of Clauses 11 through 13, wherein the one or more processors are further configured to: identify a plurality of resonance frequencies of the container at different supply levels from the training data; wherein the one or more processors being configured to cause the respective emitter to emit the non-radio based transmission to the internal space comprises the one or more processors being configured to induce an acoustic wave within the container for measurement by the sensor; and compare the measurement with one or more predetermined acoustic wave amplitudes and the plurality of resonant frequencies, wherein determining the supply status based on the measurement comprises determining the supply status based on the comparing.

Clause 15. The system of any one of Clauses 11 through 13, wherein the one or more processors are further configured to: identify a light wave profile of the container from the training data; wherein the one or more processors being configured to cause the respective emitter to emit the non-radio based transmission to the internal space comprises the one or more processors being configured to induce a light wave within the container for measurement by the sensor; compare the measurement with one or more predetermined light wave amplitudes; and wherein determining the supply status based on the measurement comprises determining the supply status based on the comparing.

Clause 16. The system of any one of Clauses 11 through 15, wherein the one or more processors are further configured to: determine that the supply status satisfies the predetermined status; responsive to determining that the supply status satisfies the predetermined threshold: activate a load cell associated with the container to measure a weight associated with the determined supply status; measure the weight associated with the determined supply status; and determine that the measured weight corresponds to the determined supply status.

Clause 17. The system of any one of Clauses 11 through 16, wherein generating the notification comprises: sending a replenishment signal to an inventory system, the replenishment signal requesting a restock of the item unit; or providing a visual or auditory alert at a dispensing device associated with the container.

Clause 18. A non-transitory machine-readable medium having instructions stored thereon that, when executed by a dispensing device, causes the dispensing device to: monitor each of a plurality of containers for a change event associated with the container, the change event comprising an indication of at least one of: a fill order for the container, a dispense of an item unit stored within the container, an opening or closing of the container, sensing a user within a predetermined proximity of the container, or a predetermined time; based on an occurrence of the change event: emit a non-radio based transmission of one or more waves to an internal space within the container and for receipt by a sensor associated with the container, wherein the internal space is fashioned to store one or more item units; receive a measurement of the one or more waves from the sensor; determine a status of the container based on the measurement and training data pertaining to measurements associated with past statuses for the container; and when the status satisfies a predetermined threshold, cause an adjustment to the container or the dispensing device.

19. The non-transitory machine-readable medium of Clause 18, wherein the instructions, when executed by dispensing device, further cause the dispensing device to: identify a plurality of resonance frequencies of the container at different supply levels from the training data; wherein the dispensing device being caused to emit the non-radio based transmission of one or more waves to the internal space comprises the dispensing device being caused to induce an acoustic wave within the container for measurement by the sensor; and compare the measurement with one or more predetermined acoustic wave amplitudes and the plurality of resonant frequencies, wherein determining the status of the container based on the measurement comprises determining a supply level of an item unit within the container based on the comparing, and wherein causing the adjustment comprises generating a notification pertaining to the container and the supply level.

20. The non-transitory machine-readable medium of Clause 18 or Clause 19, wherein the instructions, when executed by dispensing device, further cause the dispensing device to: identify a light wave profile of the container from the training data; wherein the dispensing device being caused to emit the non-radio based transmission of one or more waves to the internal space comprises the dispensing device being caused to induce a light wave within the container for measurement by the sensor; compare the measurement with one or more predetermined light wave amplitudes; and wherein determining the status of the container based on the measurement comprises determining a supply level of an item unit within the container based on the comparing, and wherein causing the adjustment comprises generating a notification pertaining to the container and the supply level.

### Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable a person of ordinary skill in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. A phrase such an implementation may refer to one or more implementations and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by any claim. Furthermore, to the extent that the term “include,” “have,” or the like is used in the description, such term is intended to be inclusive in a manner similar to the term “comprise” as “comprise” is interpreted when employed as a transitional word.

1. A computer-implemented method comprising:
monitoring each of a plurality of containers for a change event associated with the container, the change event comprising an indication of at least one of: a fill order for the container, a dispense of an item unit stored within the container, an opening or closing of the container, sensing a user within a predetermined proximity of the container, or a predetermined time; and
based on an occurrence of the change event:
emitting a non-radio based transmission of one or more waves to an internal space within the container and for receipt by a sensor associated with the container, wherein the internal space is fashioned to store one or more item units;
receiving a measurement of the one or more waves from the sensor;
determining a supply status of an item unit within the container based on the measurement and training data pertaining to measurements associated with past supply statuses for the container; and
generating, when the supply status satisfies a predetermined status, a notification pertaining to the container and the supply status.

2. The computer-implemented method of claim 1, wherein monitoring each of the plurality of containers comprises:
monitoring each of a plurality of containers within a drawer of a dispensing device.

3. The computer-implemented method of claim 1, wherein the notification comprises a replenishment request for additional supply of the item unit stored within the container, the method further comprising:
sending the replenishment request to a server configured to facilitate replenishment of the item unit.

4. The computer-implemented method of claim 1, wherein the training data comprises an intended supply status and a geometry of an interior of the container.

5. The computer-implemented method of claim 1, further comprising:
identifying a plurality of resonance frequencies of the container at different supply levels from the training data;
wherein emitting the non-radio based transmission of one or more waves to the internal space comprises inducing an acoustic wave within the container for measurement by the sensor; and
comparing the measurement with one or more predetermined acoustic wave amplitudes and the plurality of resonant frequencies,
wherein determining the supply status based on the measurement comprises determining the supply status based on the comparing.

6. The computer-implemented method of claim 1, further comprising:
identifying a light wave profile of the container from the training data;
wherein emitting the non-radio based transmission of one or more waves to the internal space comprises inducing a light wave within the container for measurement by the sensor;
comparing the measurement with one or more predetermined light wave amplitudes; and
wherein determining the supply status based on the measurement comprises determining the supply status based on the comparing.

7. The computer-implemented method of claim 1, further comprising:
determining that the supply status satisfies the predetermined status; and
responsive to determining that the supply status satisfies the predetermined threshold:
activating a load cell associated with the container to measure a weight associated with the determined supply status;
measuring the weight associated with the determined supply status; and
determining that the measured weight corresponds to the determined supply status.

8. The computer-implemented method of claim 1, wherein generating the notification comprises:
sending a replenishment signal to an inventory system, the replenishment signal requesting a restock of the item unit.

9. The computer-implemented method of claim 8, further comprising:
receiving an indication that an amount of the item unit was restocked within the container; and
updating the training data based on the determined supply status and the amount.

10. The computer-implemented method of claim 1, wherein generating the notification comprises:
providing a visual or auditory alert at a dispensing device associated with the container.

11. A system comprising:
one or more containers each configured to store a supply of an item unit;
one or more sensors, each configured to measure waves transmitted to an internal space of a container of the one or more containers, wherein the internal space of the container is fashioned to store one or more item units;
one or more emitters, each configured to emit a non-radio based transmission of one or more waves to the internal space within for receipt by a respective sensor; and
one or more processors configured to:
monitor for a change event associated with a respective container, the change event comprising an indication of at least one of: a fill order for the respective container, a dispense of an item unit stored within the respective container, an opening or closing of the respective container, sensing a user within a predetermined proximity of the respective container, or a predetermined time; and
based on an occurrence of the change event:
cause a respective emitter to emit the non-radio based transmission of the one or more waves to an internal space within the respective container;
receive a measurement of the wave from a sensor associated with the respective container;
determine a supply status associated with an item unit within the respective container based on the measurement and training data pertaining to measurements associated with past supply statuses for the respective container; and
generate, when the supply status satisfies a predetermined threshold, a notification pertaining to the respective container and the supply status.

12. The system of claim 11, further comprising:
a dispensing device comprising one or more drawers configured to store the one or more containers,
wherein monitoring each of the one or more containers comprises monitoring each of a plurality of containers within the one or more drawers for the change event, and
wherein the training data comprises an intended supply status and a geometry of an interior of the respective container.

13. The system of claim 11, wherein the notification comprises a replenishment request for additional supply of the item unit stored within the respective container, the one or more processors further configured to:
send the replenishment request to a server configured to facilitate replenishment of the item unit.

14. The system of claim 11, wherein the one or more processors are further configured to:
identify a plurality of resonance frequencies of the container at different supply levels from the training data;
wherein the one or more processors being configured to cause the respective emitter to emit the non-radio based transmission to the internal space comprises the one or more processors being configured to induce an acoustic wave within the container for measurement by the sensor; and
compare the measurement with one or more predetermined acoustic wave amplitudes and the plurality of resonant frequencies,
wherein determining the supply status based on the measurement comprises determining the supply status based on the comparing.

15. The system of claim 11, wherein the one or more processors are further configured to:
identify a light wave profile of the container from the training data;
wherein the one or more processors being configured to cause the respective emitter to emit the non-radio based transmission to the internal space comprises the one or more processors being configured to induce a light wave within the container for measurement by the sensor;
compare the measurement with one or more predetermined light wave amplitudes; and
wherein determining the supply status based on the measurement comprises determining the supply status based on the comparing.

16. The system of claim 11, wherein the one or more processors are further configured to:
determine that the supply status satisfies the predetermined status; and
responsive to determining that the supply status satisfies the predetermined threshold:
activate a load cell associated with the container to measure a weight associated with the determined supply status;
measure the weight associated with the determined supply status; and
determine that the measured weight corresponds to the determined supply status.

17. The system of claim 11, wherein generating the notification comprises:
sending a replenishment signal to an inventory system, the replenishment signal requesting a restock of the item unit; or
providing a visual or auditory alert at a dispensing device associated with the container.

18. A non-transitory machine-readable medium having instructions stored thereon that, when executed by a dispensing device, causes the dispensing device to:
monitor each of a plurality of containers for a change event associated with the container, the change event comprising an indication of at least one of: a fill order for the container, a dispense of an item unit stored within the container, an opening or closing of the container, sensing a user within a predetermined proximity of the container, or a predetermined time;
based on an occurrence of the change event:
emit a non-radio based transmission of one or more waves to an internal space within the container and for receipt by a sensor associated with the container, wherein the internal space is fashioned to store one or more item units;
receive a measurement of the one or more waves from the sensor;
determine a status of the container based on the measurement and training data pertaining to measurements associated with past statuses for the container; and
when the status satisfies a predetermined threshold, cause an adjustment to the container or the dispensing device.

19. The non-transitory machine-readable medium of claim 18, wherein the instructions, when executed by dispensing device, further cause the dispensing device to:
identify a plurality of resonance frequencies of the container at different supply levels from the training data;
wherein the dispensing device being caused to emit the non-radio based transmission of one or more waves to the internal space comprises the dispensing device being caused to induce an acoustic wave within the container for measurement by the sensor; and
compare the measurement with one or more predetermined acoustic wave amplitudes and the plurality of resonant frequencies,
wherein determining the status of the container based on the measurement comprises determining a supply level of an item unit within the container based on the comparing, and
wherein causing the adjustment comprises generating a notification pertaining to the container and the supply level.

20. The non-transitory machine-readable medium of claim 18, wherein the instructions, when executed by dispensing device, further cause the dispensing device to:
identify a light wave profile of the container from the training data;
wherein the dispensing device being caused to emit the non-radio based transmission of one or more waves to the internal space comprises the dispensing device being caused to induce a light wave within the container for measurement by the sensor; and
compare the measurement with one or more predetermined light wave amplitudes;
wherein determining the status of the container based on the measurement comprises determining a supply level of an item unit within the container based on the comparing, and
wherein causing the adjustment comprises generating a notification pertaining to the container and the supply level.
